(19) United States
(12) Patent Application Publication
(10) Pub. No.: US 2023/0338653 A1
(43) Pub. Date: Oct. 26, 2023

(54) SYSTEMS AND METHODS FOR MEAL BOLUSES IN DIABETES THERAPY

(71) Applicant: Tandem Diabetes Care, Inc., San Diego, CA (US)

(72) Inventors: Richard Rueda, San Diego, CA (US); Nicholas Sherer, Escondido, CA (US); Katherine Vyvy Tran, San Diego, CA (US); Ryan Cardenas, San Diego, CA (US); Jack Corbett, San Diego, CA (US); Peter Zhao, San Diego, CA (US); Micah Stephens, San Diego, CA (US); Colin Van Meter, San Diego, CA (US); Thomas R. Ulrich, Oceanside, CA (US); Virginia S. Lu, San Diego, CA (US)

(21) Appl. No.: 18/139,391

(22) Filed: Apr. 26, 2023

Related U.S. Application Data
(60) Provisional application No. 63/334,959, filed on Apr. 26, 2022.

(51) Int. Cl. A61M 5/172; G16H 20/17

(52) U.S. Cl. CPC A61M 5/1723; G16H 20/17; A61M 2230/201

(57) ABSTRACT

Disclosed herein are apparatuses and methods that can simplify delivery of meal boluses in diabetes therapy. Rather than requiring a user to enter a number of carbohydrates for a given meal, the system can automatically provide a predetermined amount of insulin when the user indicates that a meal is going to be consumed. Providing such predetermined, fixed meal boluses provides a lower cognitive burden alternative for the mealtime experience.

### RELATED APPLICATION

The present application claims the benefit of U.S. Provisional Application No. 63/334,959, filed Apr. 26, 2022, which is hereby incorporated herein in its entirety by reference.

### TECHNICAL FIELD

The present disclosure relates generally to ambulatory infusion pumps and, more particularly, to operation of ambulatory infusion pumps in a closed-loop or semi-closed-loop fashion.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as blood glucose meters (BGMs) and continuous glucose monitoring devices (CGMs). A CGM provides a substantially continuous estimated blood glucose level through a transcutaneous sensor that estimates blood analyte levels, such as blood glucose levels, via the patient's interstitial fluid CGM systems typically consist of a transcutaneously-placed sensor, a transmitter and a monitor.

Ambulatory infusion pumps typically allow the patient or caregiver to adjust the amount of insulin or other medicament delivered, by a basal rate or a bolus, based on blood glucose data obtained by a BGM or a CGM, and in some cases include the capability to automatically adjust such medicament delivery. Some ambulatory infusion pumps may include the capability to interface with a BGM or CGM such as, e.g., by receiving measured or estimated blood glucose levels and automatically adjusting or prompting the user to adjust the level of medicament being administered or planned for administration or, in cases of abnormally low blood glucose readings, reducing or automatically temporarily ceasing or prompting the user temporarily to cease or reduce insulin administration. These portable pumps may incorporate a BGM or CGM within the hardware of the pump or may communicate with a dedicated BGM or CGM via wired or wireless data communication protocols, directly and/or via a device such as a smartphone. One example of integration of infusion pumps with CGM devices is described in U.S. Patent Publication No. 2014/0276419, which is hereby incorporated by reference herein.

As noted above, insulin or other medicament dosing by basal rate and/or bolus techniques could automatically be provided by a pump based on readings received into the pump from a CGM device that is, e.g., external to the portable insulin pump or integrated with the pump as a pump-CGM system in a closed-loop or semi-closed-loop fashion. With respect to insulin delivery, some systems including this feature can be referred to as artificial pancreas systems because the systems serve to mimic biological functions of the pancreas for patients with diabetes. Such systems are also referred to as automated insulin delivery (AID) systems.

Some AID systems primarily deliver medicament automatically based on CGM readings, but also enable users to program meal boluses. Consumption of carbohydrates in a meal causes blood glucose to rise, which can be counteracted by insulin or other medicament delivered in a meal bolus. To program a meal bolus, a user will typically enter a number of carbohydrates in a known meal about to be consumed and the system will calculate an amount of insulin to deliver to counteract the number of carbohydrates in the meal based on a stored carbohydrate ratio for the user.

### SUMMARY

Disclosed herein are apparatuses and methods that can simplify delivery of meal boluses in diabetes therapy. Rather than requiring a user to enter a number of carbohydrates for a given meal, the system can automatically provide a predetermined amount of insulin when the user indicates that a meal is going to be consumed. Providing such predetermined, fixed meal boluses provides a lower cognitive burden alternative for the mealtime experience.

In an embodiment, a system for closed loop diabetes therapy includes a pump mechanism configured to facilitate delivery of insulin to a user, a user interface and at least one processor functionally linked to the pump mechanism and the user interface. The at least one processor can be configured to receive an indication from a user through the user interface that the user is going to consume a meal. In response to the indication, the processor can cause the pump mechanism to deliver a predetermined amount of insulin to that user as a meal bolus, where the predetermined amount of the meal bolus is not determined based on an estimated number of carbohydrates in the meal. In some embodiments, the predetermined amount is a predetermined percentage of the total daily insulin for the user.

In an embodiment, a system for closed loop diabetes therapy includes a pump mechanism configured to facilitate delivery of insulin to a user, a user interface and at least one processor functionally linked to the pump mechanism and the user interface. The at least one processor can be configured to receive an indication from a user through the user interface that the user is going to consume a meal. In response to the indication, the processor can cause the pump mechanism to deliver a predetermined amount of insulin calculated based on a total daily insulin value for the user to the user as a meal bolus.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The disclosure may be more completely understood in consideration of the following detailed description of various embodiments of the disclosure in connection with the accompanying drawings, in which:

FIG. 1 is a medical device that can be used with embodiments of the disclosure.

FIG. 2 is a block diagram representing a medical device that can be used with embodiments of the disclosure.

FIGS. 3A-3B depict an embodiment of a pump system according to the disclosure.

FIG. 4 is a schematic representation of a system according to the disclosure.

FIG. 5 depicts a user interface of an infusion pump according to the disclosure.

FIG. 6 depicts a user interface of a remote control device according to the disclosure.

FIGS. 7A-7B depict Bolus Calculator user interfaces according to the disclosure.

FIG. 8 is a flowchart of a method of delivering medicament according to an embodiment.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the disclosure.

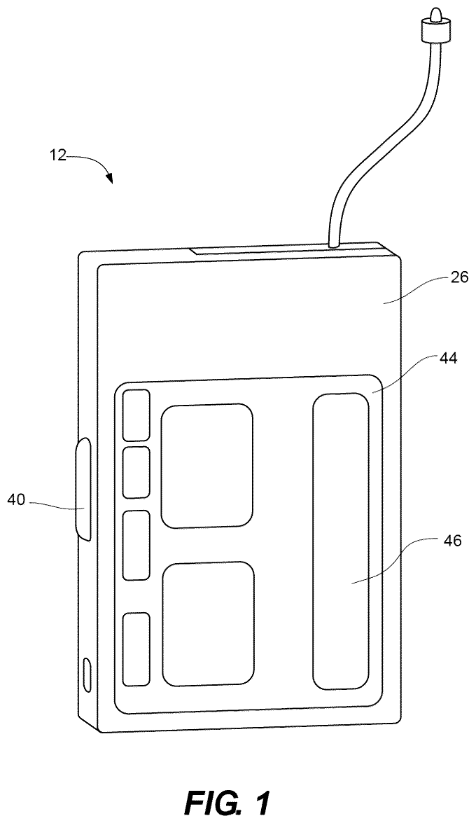

FIG. 1 depicts an embodiment of a medical device according to the disclosure. In this embodiment, the medical device is configured as a pump 12. Pump 12 may be an infusion pump that includes a pumping or delivery mechanism and reservoir for delivering medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display, a remote control device, a laptop computer, personal computer, tablet computer, a mobile communication device such as a smartphone, a wearable electronic watch or electronic health or fitness monitor, or personal digital assistant (PDA), a CGM display etc.

In one embodiment, the medical device can be an ambulatory insulin pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

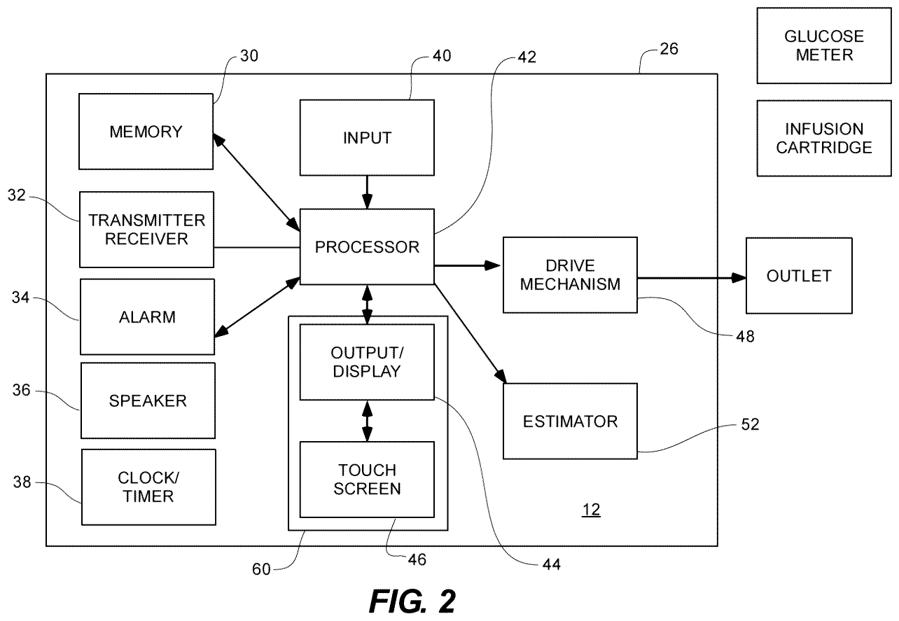

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from an input device, such as a sensor that may sense pressure, temperature or other parameters.

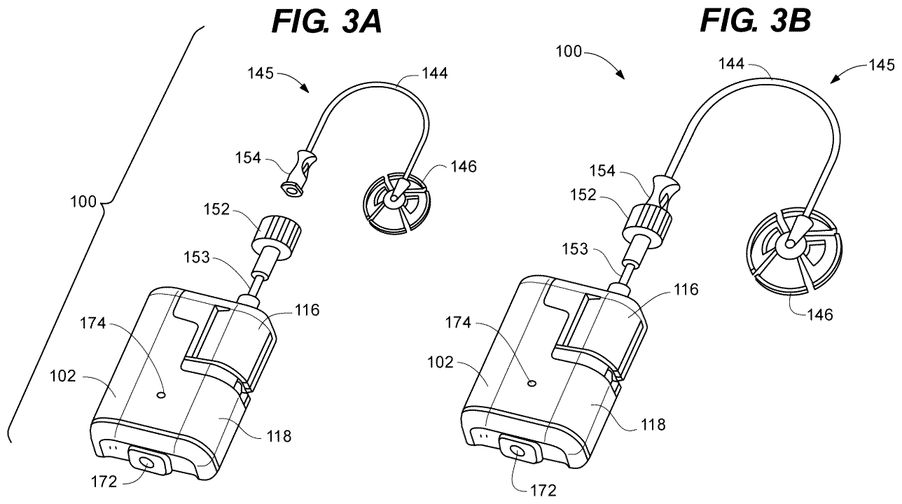

FIGS. 3A-3B depict another pump system including a pump 102 that can be used with embodiments. Drive unit 118 of pump 102 includes a drive mechanism 122 that mates with a recess in disposable cartridge 116 of pump 102 to attach the cartridge 116 to the drive unit 118. Pump system 100 can further include an infusion set 145 having a connector 154 that connects to a connector 152 attached to pump 102 with tubing 153. Tubing 144 extends to a site connector 146 that can attach or be pre-connected to a cannula and/or infusion needle that punctures the patient's skin at the infusion site to deliver medicament from the pump 102 to the patient via infusion set 145. In some embodiments, pump can include a user input button 172 and an indicator light 174 to provide feedback to the user.

In one embodiment, pump 102 includes a processor that controls operations of the pump and, in some embodiments, may receive commands from a separate device for control of operations of the pump. Such a separate device can include, for example, a dedicated remote control or a smartphone or other consumer electronic device executing an application configured to enable the device to transmit operating commands to the processor of pump 102. In some embodiments, processor can also transmit information to one or more separate devices, such as information pertaining to device parameters, alarms, reminders, pump status, etc. In one embodiment pump 102 does not include a display but may include one or more indicator lights 174 and/or one or more input buttons 172. Pump 102 can also incorporate any or all of the features described with respect to pump 12 in FIG. 2. Further details regarding such pumps can be found in U.S. Pat. No. 10,279,106 and U.S. Patent Publication Nos. 2016/0339172 and 2017/0049957, each of which is hereby incorporated herein by reference in its entirety.

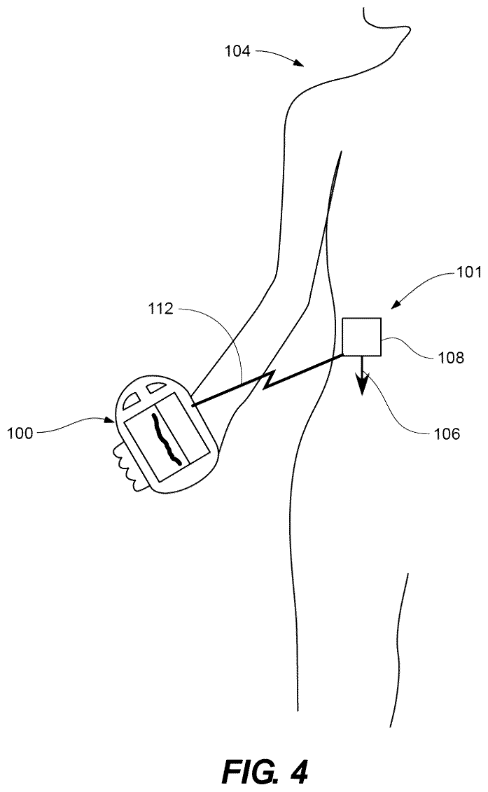

Pump 12 or 102 can interface directly or indirectly (via, e.g., a smartphone or other device) with a glucose meter, such as a blood glucose meter (BGM) or a continuous glucose monitor (CGM). Referring to FIG. 4, an exemplary CGM system 100 according to an embodiment of the present invention is shown (other CGM systems can be used). The illustrated CGM system includes a sensor 101 affixed to a patient 104 that can be associated with the insulin infusion device in a CGM-pump system. The sensor 101 includes a sensor probe 106 configured to be inserted to a point below the dermal layer (skin) of the patient 104. The sensor probe 106 is therefore exposed to the patient's interstitial fluid or plasma beneath the skin and reacts with that interstitial fluid to produce a signal that can be associated with the patient's blood glucose (BG) level. The sensor 101 includes a sensor body 108 that transmits data associated with the interstitial fluid to which the sensor probe 106 is exposed. The data may be transmitted from the sensor 101 to the glucose monitoring system receiver 100 via a wireless transmitter, such as a near field communication (NFC) radio frequency (RF) transmitter or a transmitter operating according to a “Wi-Fi” or Bluetooth® protocol, Bluetooth® low energy protocol or the like, or the data may be transmitted via a wire connector from the sensor 101 to the monitoring system 100. Transmission of sensor data to the glucose monitoring system receiver by wireless or wired connection is represented in FIG. 4 by the arrow line 112. Further detail regarding such systems and definitions of related terms can be found in, e.g., U.S. Pat. Nos. 8,311,749, 7,711,402 and 7,497,827, each of which is hereby incorporated by reference in its entirety.

In an embodiment of a pump-CGM system having a pump 12, 102 that communicates with a CGM and that integrates CGM data and pump data as described herein, the CGM can automatically transmit the glucose data to the pump. The pump can then automatically determine therapy parameters and deliver medicament based on the data. Such an automatic pump-CGM system for insulin delivery can be referred to as an automated insulin delivery (AID) or an artificial pancreas system that provides closed-loop therapy to the patient to approximate or even mimic the natural functions of a healthy pancreas. In such a system, insulin doses are calculated based on the CGM readings (that may or may not be automatically transmitted to the pump) and are automatically delivered to the patient at least in part based on the CGM reading(s). In various embodiments, doses can be delivered as automated correction boluses and/or automated increases or decreases to a basal rate. Insulin doses can also be administered based on current glucose levels and/or predicted future glucoses levels based on current and past glucose levels.

For example, if the CGM indicates that the user has a high blood glucose level or hyperglycemia, the system can automatically calculate an insulin dose necessary to reduce the user's blood glucose level below a threshold level or to a target level and automatically deliver the dose. Alternatively, the system can automatically suggest a change in therapy upon receiving the CGM data such as an increased insulin basal rate or delivery of a bolus, but can require the user to accept the suggested change prior to delivery rather than automatically delivering the therapy adjustments.

If the CGM data indicates that the user has a low blood glucose level or hypoglycemia, the system can, for example, automatically reduce a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the user, e.g., ingest carbohydrates and/or take other actions and/or make other suggestions as may be appropriate to address the hypoglycemic condition, singly or in any desired combination or sequence. Such determination can be made by the infusion pump providing therapy or by a separate device that transmits therapy parameters to the infusion pump. In some embodiments, multiple medicaments can be employed in such a system as, for example, a first medicament, e.g., insulin, that lowers blood glucose levels and a second medicament, e.g., glucagon, that raises blood glucose levels.

As with other parameters related to therapy, such thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

Automated insulin delivery (AID) systems such as those described above can also enable a user to manually program meal boluses of insulin or other medicaments to counteract the rise in blood glucose caused by the consumption of carbohydrates. Typically, a user will estimate the number of carbohydrates in a given meal and the system will calculate an amount of insulin to cover the number of carbohydrates based on a stored carbohydrate ratio for the user. However, some users may not typically deliver meal boluses, may be new to using a pump and not be familiar with counting carbohydrates for programming meal boluses and/or may sometimes be too busy to take the time to calculate the number of carbohydrates in a meal. In such circumstances, systems and methods disclosed herein can provide an improved mealtime experience that gives users a simpler alternative to a traditional meal bolus that does not require carbohydrate counting and enables a user to more quickly bolus for a meal with a lower cognitive burden.

This simpler bolus solution, which may be referred to herein as Easy Bolus, executes an algorithm that rather than requiring the user to estimate the carbohydrates in a given meal, assumes that the meal requires a predetermined amount of insulin. In embodiments, this predetermined amount is a fixed percentage of the user's Total Daily Insulin (TDI). The user's TDI can be preprogrammed into memory or can be determined based on the user's insulin delivery history with the closed loop algorithm. In embodiments employing the latter approach, the TDI for the user can therefore be regularly and/or continually updated after each day. For example, the algorithm can use the average TDI over a previous time period, such as, e.g., 7 days, with the average TDI updated after each day. The algorithm can further incorporate logic to aid in glucose stability when the fixed percentage is not optimal. Further, by providing such fixed boluses in the context of a closed loop algorithm that automatically modifies insulin delivery based on the user's CGM data, over-delivery or under-delivery of insulin for the meal can be compensated for by the algorithm automatically modifying future insulin delivery based on glucose levels of the user.

In embodiments, the fixed percentage for the Easy Bolus feature can be between 8%-11% of TDI, such as 9% of the user's TDI. In various embodiments, the percentage can be between 2%-14% of TDI. In embodiments, if the algorithm predicts that the user's glucose level will be below a predetermined threshold, such as, for example, 70 mg/dL within a predetermined period of time (i.e., in the next hour), the fixed bolus size can be reduced. It has been found that use of such an Easy Bolus feature, while not as effective as timely administration of a traditional bolus with an accurate estimate of carbs, can still significantly increase time in range relative to no bolus by 14% (using 9% TDI as the fixed percentage), thus providing a user-friendly option that can improve time in range with, e.g., a single button press compared to not bolusing. Such a feature provides an improved option any time a user is willing to compromise optimal glucose control for a quicker solution with a lower cognitive burden.

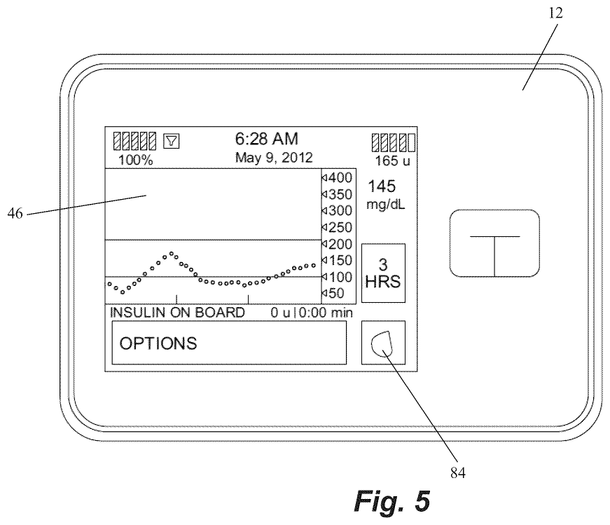

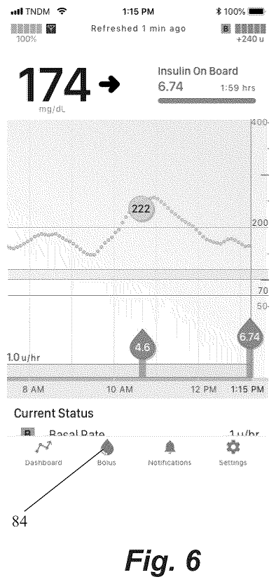

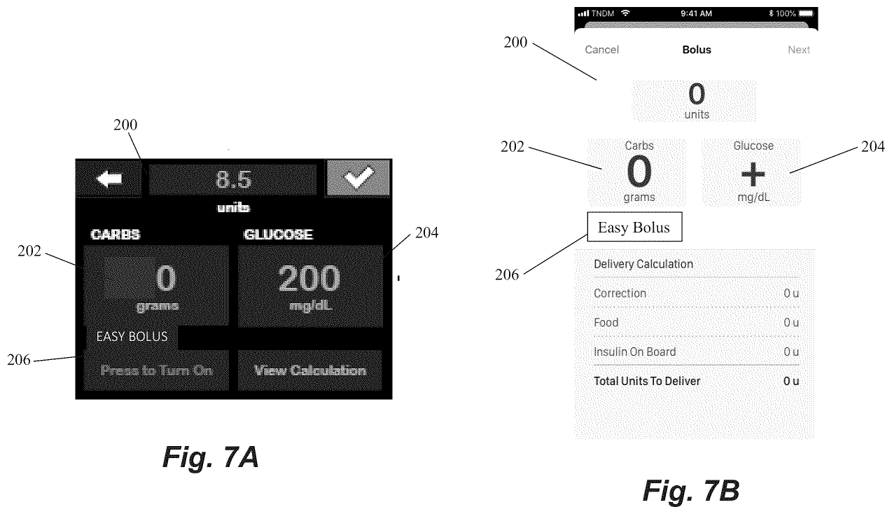

In embodiments, the Easy Bolus feature can be employed within a bolus calculator for an infusion pump executing on the pump itself and/or on a remote control device configured to control the pump. For example, after a user selects a bolus object 84 on a user interface, such as a touchscreen 46 as depicted in FIG. 5 of a pump 12 or other device for controlling a pump (FIG. 6), a Bolus Calculator page 200 can be displayed as depicted, for example, in FIG. 7A (corresponding in one embodiment to a page of a pump) and 7B (corresponding in one embodiment to a page of a device for controlling a pump). In some embodiments, the bolus calculator can include, in addition to entries for carbohydrates 202 and glucose level 204, a selectable “Easy Bolus” icon 206 that enables the user to deliver the fixed Easy Bolus in place of entering carbohydrates to calculate a meal bolus. Selection of the Easy Bolus icon 206 can cause the system to calculate a bolus using the standard bolus calculator with modified inputs. In some embodiments, the simplified bolus can be delivered automatically upon selection of the Easy Bolus option such that the bolus can be delivered with only a single button press. In other embodiments, the user can further be required to confirm the Easy Bolus following the single button press. With both options, a user who, e.g., doesn't have time to program a standard bolus, can very quickly deliver a bolus for a meal to provide better glycemic outcomes than not bolusing.

The standard bolus calculation determines the volume of the bolus delivery as Vdeliver=VAdjMeal+VAdjCorrection, where VAdjMeal is the meal bolus component of the bolus and VAdjCorrection is an optional correction bolus component based on the user's current or predicted future glucose level. In various embodiments, the glucose value may be automatically populated from a CGM reading or may be manually entered by the user. When Easy Bolus is selected, instead of using carbohydrates entered by the user to calculate the meal component, the algorithm uses VAdjMeal=xTDlest, where x is the predetermined percentage discussed above. The amount of carbohydrates intended to be addressed by the Easy Bolus algorithm can be calculated as Carbs=VAdjMeal/Carb Ratio.

In embodiments, selection of the Easy Bolus feature can also modify the glucose correction component of the bolus. For example, some systems use the glucose values of the user from the CGM to base insulin delivery calculations on a predicted glucose level of the user 30 minutes into the future, or gPred30. In Easy Bolus mode, the system can look further forward into the future, such as, for example, 1 hour further, or gPred90. In embodiments, this prediction can be based on the rate of change of gPred30 per hour such that gPred90=gPred30(t)+rate per hour, in which the current gPred30 value and the gPred30 value one hour in the past are used to calculate rate per hour=gPred30(t)—gPred30(t−60) (such that consolidating the equation determines gPred90=2×gPred30−gPred30(t−60)). In some embodiments, the maximum bolus size that can be delivered with the Easy Bolus feature can be set as xTDlest such that the correction component of the bolus based on the glucose level of the user (i.e., gPred90) cannot be used to increase the amount of the Easy Bolus and can only decrease the amount by applying a negative correction. In some embodiments, a negative correction is applied only when GPred90 is below 70 mg/dL (i.e., indicates potential future hypoglycemia if the full Easy Bolus is delivered). In such embodiments, the system has determined that the predetermine amount of the Easy Bolus is not optimal, and accordingly makes a downward adjustment to the amount to reduce the risk of hypoglycemia.

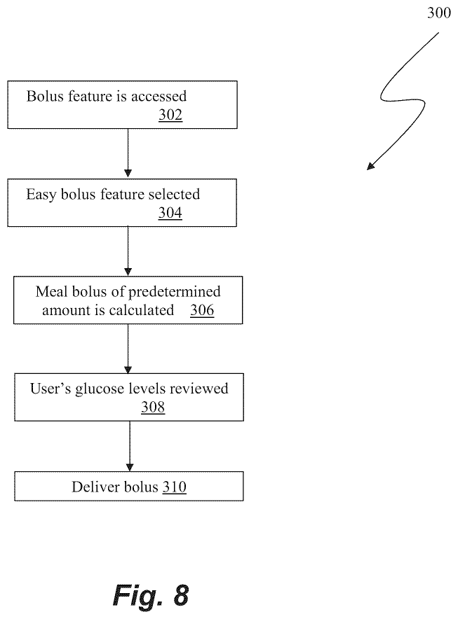

Referring now to FIG. 8, a flowchart of steps in a method of diabetes therapy 300 is depicted. At step 302, a bolus calculator feature of an infusion pump system is accessed by a user. An Easy Bolus feature provided within the bolus calculator is selected at step 304. A meal bolus of a predetermined amount is calculated at step 306, rather than being calculated based on a specific estimated number of carbohydrates expected to be consumed. As noted above, this predetermined amount may be calculated as a predetermined percentage of the user's total daily insulin amount. At step 308, the user's glucose levels are reviewed for potential modification to the calculated bolus. As described herein, the user's glucose level 90 minutes in the future is calculated and if the value is below 70 mg/dL, the bolus amount can be reduced. At step 310, the bolus is delivered to the user.

Significant simulations of patient therapy were conducted comparing Easy Bolus to not bolusing. It was found that use of Easy Bolus increased Time in Range (i.e., the time the user's glucose levels are within the user's target range) compared to not bolusing without significantly increasing low glucose levels (i.e., time below 70 mg/dL). For example, it was found that an Easy Bolus of 9% of the user's TDI increased time in range by 14% compared to not bolusing with only a 0.4% increase in time below 70 mg/dL. In addition, while delivery of a standard bolus calculated based on a number of carbohydrates estimated for the specific meal will have a higher time in range than Easy Bolus, it was further found the use of a 9% Easy Bolus will decrease the time below 70 mg/dL by 0.5% compared to a standard bolus. It was further found that Easy Bolus has greater effectiveness for small to medium sized meals (i.e., 5% to 20% of daily carbs) compared to larger meals (i.e., over 40% of daily carbs) such that in some embodiments users can be recommended and/or instructed to generally only employ Easy Bolus with small to medium sized meals.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; and 11,464,908 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0154405; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677 and 2023/0047034 and commonly owned U.S. patent application Ser. Nos. 17/368,968; 17/896,492; 17/961,206; 17/964,513; 18/011,060; 18/071,814; 18/071,835; 18/075,029; and Ser. No. 18/115,316.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A system for closed loop diabetes therapy, comprising:
a pump mechanism configured to facilitate delivery of insulin to a user;
a user interface;
at least one processor functionally linked to the pump mechanism and the user interface, the at least one processor configured to:
receive an indication from a user through the user interface that the user is going to consume a meal; and
cause the pump mechanism to deliver a predetermined amount of insulin to the user as a meal bolus in response to the indication that the user is going to consume a meal, wherein the predetermined amount of the meal bolus is not determined based on an estimated number of carbohydrates in the meal.

2. The system of claim 1, wherein the predetermined amount of insulin is based on a total daily insulin value for the user.

3. The system of claim 2, wherein the predetermined amount of insulin is a fixed percentage of the total daily insulin value.

4. The system of claim 2, further comprising a memory and wherein the total daily insulin value for the user is preprogrammed into the memory.

5. The system of claim 2, wherein the total daily insulin value on insulin delivery history for the user.

6. The system of claim 5, wherein the total daily insulin value for the user is updated at regular intervals based on subsequent insulin delivery.

7. The system of claim 1, wherein the at least one processor is further configured to determine if the user's glucose level is predicted to be below a low glucose threshold within a predetermined period of time of receiving the indication that the user is going to consume the meal and, if so, to reduce the predetermined amount.

8. The system of claim 1, wherein the at least one processor is configured to display a bolus calculator on the user interface that enables a user to enter one or more of a glucose level or a number of carbohydrates for calculation of the bolus.

9. The system of claim 8, wherein the at least one processor to display an option on the bolus calculator that will cause the predetermined amount to be delivered without entering a glucose level or a number of carbohydrates.

10. The system of claim 9, wherein the indication that the user is going to consume the meal is selection of the option.

11. A system for closed loop diabetes therapy, comprising:
a pump mechanism configured to facilitate delivery of insulin to a user;
a user interface;
at least one processor functionally linked to the pump mechanism and the user interface, the at least one processor configured to:
cause the pump mechanism to deliver a predetermined amount of insulin to the user as a meal bolus in response to the indication that the user is going to consume a meal; and
deliver a predetermined amount of insulin calculated based on a total daily insulin value for the user to the user as a meal bolus.

12. The system of claim 11, wherein the predetermined amount of insulin is a fixed percentage of the total daily insulin value.

13. The system of claim 11, further comprising a memory and wherein the total daily insulin value for the user is preprogrammed into the memory.

14. The system of claim 11, wherein the total daily insulin value on insulin delivery history for the user.

15. The system of claim 14, wherein the total daily insulin value for the user is updated at regular intervals based on subsequent insulin delivery.

16. The system of claim 11, wherein the at least one processor is further configured to determine if the user's glucose level is predicted to be below a low glucose threshold within a predetermined period of time of receiving the indication that the user is going to consume the meal and, if so, to reduce the predetermined amount.

17. The system of claim 11, wherein the at least one processor is configured to display a bolus calculator on the user interface that enables a user to enter one or more of a glucose level or a number of carbohydrates for calculation of the bolus.

18. The system of claim 17, wherein the at least one processor to display an option on the bolus calculator that will cause the predetermined amount to be delivered without entering a glucose level or a number of carbohydrates.

19. The system of claim 17, wherein the indication that the user is going to consume the meal is selection of the option.
